(19) United States
(12) Patent Application Publication
(10) Pub. No.: US 2025/0102501 A1
(43) Pub. Date: Mar. 27, 2025

(54) SYSTEMS AND METHODS FOR MEMBRANE-BASED ASSAYING

(71) Applicant: Solaris BioSciences, Inc., Providence, RI (US)

(72) Inventor: Nabil Lawandy, Saunderstown, RI (US)

(21) Appl. No.: 18/729,959

(22) PCT Filed: Jan. 18, 2023
(86) PCT No.: PCT/US2023/011050

§ 371 Date: Jul. 18, 2024

Related U.S. Application Data
(60) Provisional application No. 63/300,337, filed on Jan. 18, 2022.

(51) Int. Cl. G01N 33/543; G01N 21/64

(52) U.S. Cl. CPC G01N 33/54386; G01N 21/6428; G01N 21/645; G01N 2021/6471

(57) ABSTRACT

A system and associated method for determining the presence of a reactive compound in an analyte, including a membrane having a plurality of channels or pores having surfaces affixed with one or more of functionalized nanoporous materials, antibodies, and proteins capable of reacting with the reactive compound in the analyte and changing the porosity of the membrane from a first porosity to a second porosity; a fluid source for contacting the membrane with a fluid; a gasochromic material configured such that a portion of the fluid in contact with the membrane flows through the plurality of channels or pores and contacts the gasochromic material; an excitation source capable of exciting the gasochromic material to generate an emission; a detection device capable of detecting the emission; and a processor configured to process data obtained by the detection device corresponding to the first porosity or the second porosity of the membrane.

### CROSS-REFERENCE TO RELATED APPLICATIONS

The present application claims priority to U.S. provisional application Ser. No. 63/300,337, filed Jan. 18, 2022, the disclosure of which is incorporated herein by reference in its entirety.

### TECHNICAL FIELD

The present invention relates generally to systems and methods for determining the presence of a desired compound through a membrane-based assay. More specifically, the present invention relates to the use of membranes having surface-functionalized channels or pores for determining the presence of a desired compound in a fluid disposed in contact with the membrane.

### BACKGROUND OF THE INVENTION

Biomolecules such as proteins, antibodies, DNA strands, red blood cells and semen, molecular and biological moieties, and large molecules in general, are commonly detected and separated using electrophoresis in gels and other media. Electrophoresis is the motion of dispersed particles relative to a fluid under the influence of a spatially uniform electric field. This electrokinetic phenomenon was observed for the first time in 1807 by Ferdinand Frederic Reuss (Moscow State University), who noticed that the application of a constant electric field caused clay particles dispersed in water to migrate. Electrophoresis is ultimately caused by the presence of a charged interface between the particle surface and the surrounding fluid, and it is the basis for a number of analytical techniques used in biochemistry for separating molecules by size, charge or binding affinity.

Electrophoresis is a technique used in laboratories to separate macromolecules based on their size. The technique involves applying a negative charge so that particles such as proteins move toward a positive charge. This technique is used for both DNA and RNA analysis. Polyacrylamide gel electrophoresis (PAGE) has a clearer resolution than electrophoresis based in agarose and is more suitable for quantitative analysis. Using PAGE, DNA foot-printing can identify how proteins bind to DNA. PAGE can be used to separate proteins by size, density and purity, and further may be used for plasmid analysis for developing an understanding of bacteria becoming resistant to antibiotics.

Recently, dielectrophoresis (DEP), which uses electric field gradients, has been utilized for similar applications and cell separation. DEP does not require that the macromolecules be charged, and instead relies on the polarizability of the macromolecules. Dielectrophoresis occurs when a polarizable particle is suspended in a non-uniform electric field. The particles are polarized in the electric field, and the particles' poles experience a force along the field lines, which force can be either attractive or repulsive, according to the orientation of the dipole. Since the field is non-uniform, the pole experiencing the greater electric field will dominate the other, and the particle will move. The orientation of the dipole is dependent on the relative polarizability of the particle and medium, in accordance with Maxwell-Wagner-Sillars polarization. Further, since the direction of the force is dependent on field gradient rather than field direction, dielectrophoresis will occur in alternating current as well as direct current electric fields; polarization, and hence the direction of the force, will depend on the relative polarizabilities of particle and medium. If the particle moves in the direction of increasing electric field, the behavior is referred to as positive DEP. If acting to move the particle away from high field regions, it is known as negative DEP (nDEP). As the relative polarizabilities of the particle and medium are frequency dependent, varying the energizing signal and measuring the manner in which the force changes can be used to determine the electrical properties of particles; this allows for the elimination of electrophoretic motion of particles due to inherent particle charge.

Additional phenomena associated with dielectrophoresis are electrorotation and traveling wave dielectrophoresis (TWDEP). These require complex signal generation equipment and patterned electrode structures to create the required rotating or traveling electric fields. As a result of this complexity, these techniques have found less favor than conventional dielectrophoresis among researchers.

In addition to electrophoretic separation, identification and separation is accomplished by methods of attaching proteins or molecule-specific fluorescent or chemiluminescent markers used in the Southern Blot and Western Blot assays to identify electrophoretically separated macromolecules obtained from the lysing of cells. The techniques of electrophoresis and blotting are capable of handling biological moieties ranging from 10-1000 kD, or 3-100 nanometers along various dimensions. However, there are several shortcomings associated with these technologies, including a one-day performance cycle duration, being limited to charged species, difficulty handling large molecules (e.g., titin), a requirement of milliliter samples and several reagents, and an inability to produce information regarding dielectric properties. More recently, nonlinear four-wave mixing techniques have been employed to identify specific molecules in conjunction with electrophoretic or dielectrophoretic separation. For example, ultrasensitive detection of proteins and antibodies by absorption-based laser wave-mixing detection using a chromophore label has been demonstrated by Tong et al. The four-wave mixing signal results in an absorption grating formed by the linkage of a non-fluorescing chromophore label, Coomassie Brilliant Blue (CBB), which absorbs the laser radiation.

In light of the aforementioned deficiencies associated with known detection/separation techniques, there exists a need for superior means of identifying compounds in an analyte.

### SUMMARY OF THE INVENTION

In general, in one aspect, the invention features a system for determining the presence of a reactive compound in an analyte, including a membrane having a first porosity, the membrane having a first side, a second side opposite from the first side, and a plurality of channels or pores having surfaces affixed with one or more of functionalized nanoporous materials, antibodies, and proteins capable of reacting with the reactive compound in the analyte when the analyte is disposed in contact with the membrane and changing the porosity of the membrane from the first porosity to a second porosity; a fluid source directed at the first side of the membrane for contacting the membrane with a fluid; a gasochromic material disposed adjacent to the second side of the membrane and configured such that a portion of the fluid in contact with the membrane flows through the plurality of channels or pores and contacts the gasochromic material; an excitation source directed at the gasochromic material and capable of exciting the gasochromic material to generate an emission; a detection device directed at the gasochromic material and capable of detecting the emission generated from excitation of the gasochromic material; and a processor connected to the detection device and configured to process data obtained by the detection device corresponding to the first porosity or the second porosity of the membrane, where the emission is quenched by the fluid that contacts the gasochromic material.

Implementations of the invention may include one or more of the following features. The analyte may include blood plasma, blood, urine, ocular fluid, or spinal fluid. The reactive compound may be a protein, an antibody, a DNA strand, a red blood cell, semen, or a molecular or biological moiety. The membrane may include polytetrafluoroethylene (PTFE), polyethylene terephthalate (PET), polystyrene (PS), an acrylic, a polysulfone polymer, graphene, a graphene oxide, or a hydrophilic semiconductor. The fluid source may include one or more of a fluid pump, a line gas source, a dispensing outlet, and a solenoid valve. The fluid may include water, oxygen gas (O2), argon gas (Ar), helium gas (He), xenon gas (Xe), or nitrogen gas (N2). The gasochromic material may include platinum, rhodium, platinum-containing porophyrines, iridium-containing phosphyrines, nanocrystalline zinc-oxide, or polystyrene (PS). The excitation source may be a light-emitting diode (LED), a lamp, or a laser. The detection device may include one or more of an imaging device and a sensor. The detection device may include one or more filters configured to filter out all radiation except for the emission generated from excitation of the gasochromic material.

In general, in another aspect, the invention features a method for determining the presence of a reactive compound in an analyte, including providing a membrane having a first porosity, the membrane having a first side, a second side opposite from the first side, and a plurality of channels or pores having surfaces affixed with one or more of functionalized nanoporous materials, antibodies, and proteins capable of reacting with the reactive compound in the analyte when the analyte is disposed in contact with the membrane and changing the porosity of the membrane from the first porosity to a second porosity; disposing a fluid source in a position directed at the first side of the membrane and a gasochromic material in a position adjacent to the second side of the membrane; contacting the membrane with the analyte; contacting the membrane with a fluid from the fluid source such that a portion of the fluid in contact with the membrane flows through the plurality of channels or pores and contacts the gasochromic material; exciting, by an excitation source directed at the gasochromic material, the gasochromic material to generate an emission; detecting, by a detection device directed at the gasochromic material, the emission generated from excitation of the gasochromic material; and processing, by a processor connected to the detection device, data obtained by the detection device corresponding to the first porosity or the second porosity of the membrane, where the emission is quenched by the fluid that contacts the gasochromic material.

Implementations of the invention may include one or more of the following features. The analyte may include blood plasma, blood, urine, ocular fluid, or spinal fluid. The reactive compound may be a protein, an antibody, a DNA strand, a red blood cell, semen, or a molecular or biological moiety. The membrane may include polytetrafluoroethylene (PTFE), polyethylene terephthalate (PET), polystyrene (PS), an acrylic, a polysulfone polymer, graphene, a graphene oxide, or a hydrophilic semiconductor. The fluid source may include one or more of a fluid pump, a line gas source, a dispensing outlet, and a solenoid valve. The fluid may include water, oxygen gas (O2), argon gas (Ar), helium gas (He), xenon gas (Xe), or nitrogen gas (N2). The gasochromic material may include platinum, rhodium, platinum-containing porophyrines, iridium-containing phosphyrines, nanocrystalline zinc-oxide, or polystyrene (PS). The excitation source may be a light-emitting diode (LED), a lamp, or a laser. The detection device may include one or more of an imaging device and a sensor. The detection device may include one or more filters configured to filter out all radiation except for the emission generated from excitation of the gasochromic material.

### BRIEF DESCRIPTION OF THE DRAWINGS

The above-mentioned and other aspects, features and advantages can be more readily understood from the following detailed description with reference to the accompanying drawings, wherein:

FIG. 1 is an illustration of an exemplary membrane of the present invention; and

FIG. 2 is a diagram of a system and associated methodology for determining the porosity of a membrane according to an embodiment of the present invention.

### DETAILED DESCRIPTION OF THE PREFERRED EMBODIMENTS

The present invention provides for systems and methods for determining the presence of a desired compound through a membrane-based assay. More specifically, the present invention provides for systems and methods of using membranes having surface-functionalized channels or pores for determining the presence of a desired compound in a fluid disposed in contact with the membrane.

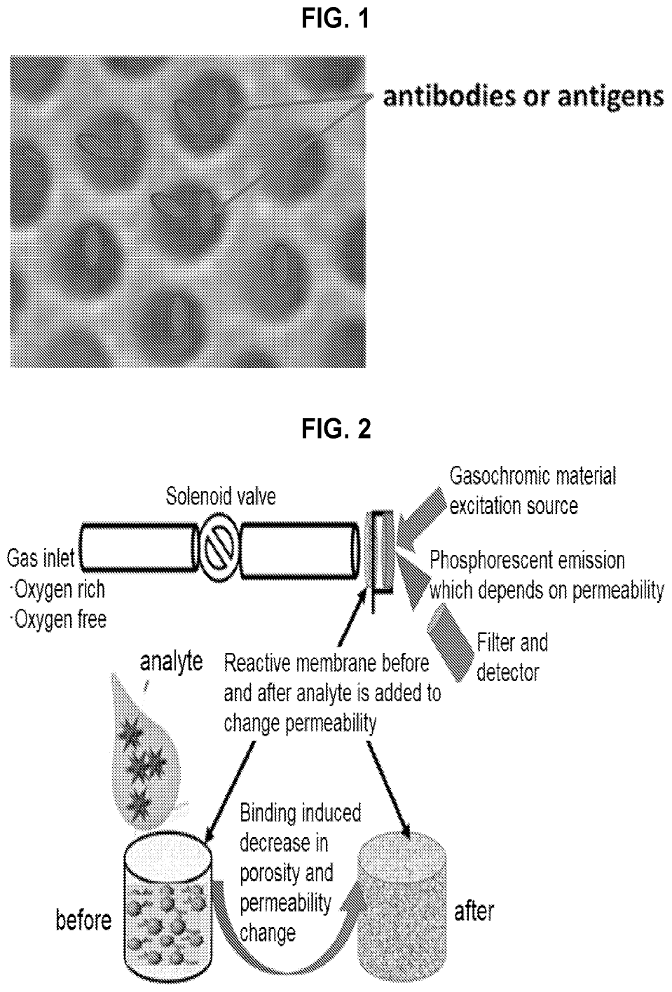

A membrane of the present invention may include but is not limited to membranes including functionalized nanoporous material(s) and membranes including antibodies, proteins, and the like disposed in and attached to the surfaces of membrane channels or pores. FIG. 1 provides an illustrative embodiment of a membrane of the present invention, namely a membrane having channels or pores therein, where the surfaces of these channels or pores contain reactive groups disposed thereon and attached thereto, the reactive groups including antibodies and/or antigens.

The membranes of the present invention may be configured such that, in response to wetting of the membrane with a fluid, or analyte, including the desired compound(s) (e.g., protein or moiety of interest), the desired compound(s) react with the binding compound(s) (e.g., antibody) disposed in and attached to the surface of the membrane channel or pore. The result of such binding is a reduction in pore size, i.e., a porosity change. This reduction in pore size or porosity change may be utilized as an assay for the desired compound. The desired compound may be any compound of interest, including but not limited to a protein, an antibody, a DNA strand, a red blood cell, semen, or a molecular or biological moiety. Additionally, the analyte may be any acceptable fluid, including but not limited to blood plasma, blood, urine, ocular fluid, or spinal fluid.

Membrane materials include but are not limited to polytetrafluoroethylene (PTFE), polyethylene terephthalate (PET), polystyrene (PS), acrylics, polysulfone polymers, graphene, graphene oxides, and semiconductors, particularly those with hydrophilic properties in embodiments in which the wetting fluid is water.

A methodology of the present invention for determining the presence of a desired compound includes wetting or otherwise disposing a fluid in contact with a membrane of the present invention, washing the membrane to carry away or otherwise remove all moieties not selectively attached to the relevant sites (e.g., membrane channels or pores), drying the membrane (optionally under mild heat), and measuring permeability (e.g., gas permeability) of the membrane as compared to permeability of the membrane prior to the aforementioned wetting and drying steps. Such comparison provides for determination as to whether the desired compound was in the membrane-contacting fluid.

FIG. 2 provides a system and associated methodology for determining the porosity of a membrane according to an embodiment of the present invention. An analyte is disposed in contact with the membrane for purposes of determining whether the analyte includes the desired compound. Should the desired compound be present in the analyte, the compound will bind to the binding compound(s) of the membrane (e.g., those attached to the surface of the membrane channel or pore), resulting in a binding-induced decrease in porosity and/or permeability change.

The system may include a fluid container, a fluid dispenser, and/or a fluid source. The fluid source may be any device known to those skilled in the art that is configured to dispense, direct, and/or control the flow of a fluid (i.e., a liquid or a gas) including, but not limited to, a pump and a line gas source. The fluid source may be powered by any means known to those skilled in the art, including but not limited to, electric, hydraulic, motor, pneumatic, and manual. In addition, the fluid source may include multiple fluid dispensing outlets. Alternatively, the fluid source may include a single dispensing outlet. A solenoid valve may be provided in connection with the fluid delivery.

The fluid source may be connected to a fluid container. The fluid container may hold any fluid (i.e., liquid or gas) known to those skilled in the art that is capable of displacing an equilibrium concentration of oxygen in a gasochromic material upon contact with the gasochromic material. For example, the fluid may be any liquid or gas that is rich in oxygen, including oxygen gas (O2). Alternatively, the fluid may be any liquid or gas that contains substantially no oxygen, including, but not limited to argon, helium, xenon, and nitrogen, including nitrogen gas (N2). A nitrogen generator may be utilized in connection with the present invention for providing the desired nitrogen gas as well as disinfection, such nitrogen generator may be configured to generate nitrogen from an air input through membrane separation or pressure swing adsorption (PSA).

As previously discussed, the fluid may be capable of displacing the equilibrium concentration of oxygen in the gasochromic material, illustrated in red in the embodiment of FIG. 2. The gasochromic material may be any material configured to change the intensity or spectral position of its emission or absorption bands in response to various molecular moieties. For example, the gasochromic material may be any desired low molecular weight polymer material known to those skilled in the art that contains gasochromic molecules. The gasochromic molecules may be any molecules configured to emit light under excitation by UV light or other wavelengths including, but not limited to, platinum, rhodium, platinum-containing porophyrines, and iridium-containing phosphyrines and nanocrystalline zinc-oxide. For example, in one embodiment, the gasochromic material may be a low molecular weight polymer coating, such as polystyrene (PS), containing gasochromic molecules. Alternatively, the gasochromic material may be a film, such as polystyrene (PS), containing gasochromic molecules.

In an alternative embodiment, the gasochromic material may be mounted on a substrate. The substrate may be any substrate configured to maintain the gasochromic material in a desired position and configured to enable a detection device to sense light, radiation, or other emission emitted from the gasochromic material when the fluid contacts the gasochromic material. In one embodiment, the substrate may be a transparent substrate. In alternative embodiments, the substrate may be doped with gasochromic moieties.

The gasochromic material may be configured to emit light, radiation, or other emission under excitation. FIG. 2 illustrates that excitation of the gasochromic material may be accomplished via an excitation source. The excitation source may be any device configured to emit light, radiation, or other emission that is capable of causing the gasochromic molecules in the gasochromic material to emit a phosphorescent emission, for example, such as a phosphorescent transition from a triplet state to a singlet ground state. For example, the excitation source may be an LED or a lamp. Alternatively, the excitation source may be a laser.

When the gasochromic molecules in the gasochromic material are in an excited state, the emission from the gasochromic molecules/material may be sensed by a detection device. The detection device may be any device known to those skilled in the art that may be configured to sense emission, capture images, and/or create images. In one embodiment, for example, the detection device may include an imaging device, such as a camera. In addition, or alternatively, the detection device may include at least one sensor configured to sense the emission. The sensors may be any sensors known to those skilled in the art including, but not limited to, photodiodes, photomultipliers, and photovoltaic cells. The detection device may include one or more filters. The filter may be any device known to those skilled in the art configured to reject all light or radiation other than emission from the gasochromic molecules. For example, in one embodiment, the filter may be a Schott red glass 610 (RG 610).

The system of the present invention, including that of FIG. 2, may also include a processor known to those skilled in the art. The processor may be configured to receive the detected images from the detection devices and output porosity data based on the detected images. The porosity data may include data corresponding to the emission from the gasochromic molecules in the gasochromic material. For example, when a fluid that is rich in oxygen is dispensed to flow through the membrane, the detected emission from the gasochromic material is inversely related to the porosity of the membrane: a lower detection of emission corresponds to a higher level of porosity. Conversely, when a fluid that has substantially no oxygen is dispensed to flow through the membrane, the detected emission is directly related to the porosity of the membrane: a lower detection of emission corresponds to a lower level of porosity.

The present invention includes a method of determining the porosity of a membrane. The method may first include positioning a positioning the membrane in a space between the fluid source and the gasochromic material. The membrane may be positioned such that it may be secured between the fluid source and the gasochromic material. For example, the system may include a device configured to maintain the membrane in a substantially flat position, such as a plate. The device (i.e., plate) may also be configured to attach to the fluid source and enable the fluid source to dispense the fluid through the membrane. Alternatively, the membrane may be positioned such that the membrane may be advanced along its longitudinal axis and, thereby, movable relative to the fluid source, the gasochromic material, and the detection device.

After the membrane is positioned in the space between the fluid source and the gasochromic material, fluid may be dispensed through an outlets of the fluid source, for example, such that at least a portion of the dispensed fluid can flow through the membrane. Fluid that flows completely through the membrane may contact the gasochromic material and may quench emission of the gasochromic molecules in the gasochromic material. In particular, a portion of the dispensed fluid that flows from a side of the membrane facing the fluid source to a side of the membrane facing the gasochromic material may disperse along a width of the gasochromic material. For example, at least some of the portion of the dispensed fluid may disperse in a direction substantially perpendicular to a flow path of the fluid through the membrane.

The method further includes powering the excitation source such that the excitation source may emit UV or other wavelengths configured to excite the gasochromic molecules in the gasochromic material. The excitation source may be positioned such that at least one path of light, radiation, or emission from the excitation source intersects with the gasochromic material. In addition, the excitation source may be powered prior to, during, and after the fluid contacts the gasochromic material, so that the detection device may be capable of detecting emission of the gasochromic molecules, such as that corresponding to the equilibrium concentration of oxygen in the gasochromic material and the emission corresponding to the displaced equilibrium concentration of oxygen in the gasochromic material. Thus, the porosity of the membrane may be related to the change in the detected emission corresponding to the equilibrium concentration of oxygen in the gasochromic material and the detected emission corresponding to the displaced equilibrium concentration of oxygen in the gasochromic material.

During excitation of the gasochromic molecules in the gasochromic material, the detection device may be detecting the emitted light by first using a filter to reject all light or radiation other than the emission from the gasochromic molecules. After filtering the light or radiation, the detection device may use sensors therein to detect the emission. The detection device may further transmit the detected signals to a processor, which may be configured to determine and output data corresponding to the porosity of the relevant membrane.

The determination and output of data corresponding to the porosity of the membrane may be calculated based on an average porosity over the entire membrane. For example, the membrane may be secured between the fluid source and the gasochromic material, and the fluid source may be configured to dispense the fluid on the membrane such that a porosity determination may be made across the entire membrane. Alternatively, porosity may be determined along the length of the membrane. For example, the membrane may be positioned in a space between the fluid source and the gasochromic material. The membrane may be advanced through the space along its longitudinal axis. As the membrane is advanced through the space, the fluid source may dispense fluid along the length of the membrane such that the detection device may obtain data corresponding to the porosity of the membrane along its length.

The embodiments and examples above are illustrative, and many variations can be introduced to them without departing from the spirit of the disclosure or from the scope of the invention. For example, elements and/or features of different illustrative and exemplary embodiments herein may be combined with each other and/or substituted with each other within the scope of this disclosure. Therefore, it is intended that the invention not be limited to the particular embodiments disclosed, but that the invention will include all embodiments falling within the scope of the claims. For a better understanding of the invention, its operating advantages and the specific objects attained by its uses, reference should be had to the accompanying drawings and descriptive matter, in which there is illustrated a preferred embodiment of the invention.

What is claimed is:

1. A system for determining the presence of a reactive compound in an analyte, comprising:
a membrane having a first porosity, the membrane having a first side, a second side opposite from the first side, and a plurality of channels or pores having surfaces affixed with one or more of functionalized nanoporous materials, antibodies, and proteins capable of reacting with the reactive compound in the analyte when the analyte is disposed in contact with the membrane and changing the porosity of the membrane from the first porosity to a second porosity;
a fluid source directed at the first side of the membrane for contacting the membrane with a fluid;
a gasochromic material disposed adjacent to the second side of the membrane and configured such that a portion of the fluid in contact with the membrane flows through the plurality of channels or pores and contacts the gasochromic material;
an excitation source directed at the gasochromic material and capable of exciting the gasochromic material to generate an emission;
a detection device directed at the gasochromic material and capable of detecting the emission generated from excitation of the gasochromic material; and
a processor connected to the detection device and configured to process data obtained by the detection device corresponding to the first porosity or the second porosity of the membrane,
wherein the emission is quenched by the fluid that contacts the gasochromic material.

2. The system of claim 1, wherein the analyte includes blood plasma, blood, urine, ocular fluid, or spinal fluid.

3. The system of claim 1, wherein the reactive compound is a protein, an antibody, a DNA strand, a red blood cell, semen, or a molecular or biological moiety.

4. The system of claim 1, wherein the membrane includes polytetrafluoroethylene (PTFE), polyethylene terephthalate (PET), polystyrene (PS), an acrylic, a polysulfone polymer, graphene, a graphene oxide, or a hydrophilic semiconductor.

5. The system of claim 1, wherein the fluid source includes one or more of a fluid pump, a line gas source, a dispensing outlet, and a solenoid valve.

6. The system of claim 1, wherein the fluid includes water, oxygen gas (O₂), argon gas (Ar), helium gas (He), xenon gas (Xe), or nitrogen gas (N₂).

7. The system of claim 1, wherein the gasochromic material includes platinum, rhodium, platinum-containing porophyrines, iridium-containing phosphyrines, nanocrystalline zinc-oxide, or polystyrene (PS).

8. The system of claim 1, wherein the excitation source is a light-emitting diode (LED), a lamp, or a laser.

9. The system of claim 1, wherein the detection device includes one or more of an imaging device and a sensor.

10. The system of claim 1, wherein the detection device includes one or more filters configured to filter out all radiation except for the emission generated from excitation of the gasochromic material.

11. A method for determining the presence of a reactive compound in an analyte, comprising:
providing a membrane having a first porosity, the membrane having a first side, a second side opposite from the first side, and a plurality of channels or pores having surfaces affixed with one or more of functionalized nanoporous materials, antibodies, and proteins capable of reacting with the reactive compound in the analyte when the analyte is disposed in contact with the membrane and changing the porosity of the membrane from the first porosity to a second porosity;
disposing a fluid source in a position directed at the first side of the membrane and a gasochromic material in a position adjacent to the second side of the membrane;
contacting the membrane with the analyte;
contacting the membrane with a fluid from the fluid source such that a portion of the fluid in contact with the membrane flows through the plurality of channels or pores and contacts the gasochromic material;
exciting, by an excitation source directed at the gasochromic material, the gasochromic material to generate an emission;
detecting, by a detection device directed at the gasochromic material, the emission generated from excitation of the gasochromic material; and
processing, by a processor connected to the detection device, data obtained by the detection device corresponding to the first porosity or the second porosity of the membrane,
wherein the emission is quenched by the fluid that contacts the gasochromic material.

12. The method of claim 11, wherein the analyte includes blood plasma, blood, urine, ocular fluid, or spinal fluid.

13. The method of claim 11, wherein the reactive compound is a protein, an antibody, a DNA strand, a red blood cell, semen, or a molecular or biological moiety.

14. The method of claim 11, wherein the membrane includes polytetrafluoroethylene (PTFE), polyethylene terephthalate (PET), polystyrene (PS), an acrylic, a polysulfone polymer, graphene, a graphene oxide, or a hydrophilic semiconductor.

15. The method of claim 11, wherein the fluid source includes one or more of a fluid pump, a line gas source, a dispensing outlet, and a solenoid valve.

16. The method of claim 11, wherein the fluid includes water, oxygen gas (O₂), argon gas (Ar), helium gas (He), xenon gas (Xe), or nitrogen gas (N₂).

17. The method of claim 11, wherein the gasochromic material includes platinum, rhodium, platinum-containing porophyrines, iridium-containing phosphyrines, nanocrystalline zinc-oxide, or polystyrene (PS).

18. The method of claim 11, wherein the excitation source is a light-emitting diode (LED), a lamp, or a laser.

19. The method of claim 11, wherein the detection device includes one or more of an imaging device and a sensor.

20. The method of claim 11, wherein the detection device includes one or more filters configured to filter out all radiation except for the emission generated from excitation of the gasochromic material.
